(19) United States
(12) Patent Application Publication
(10) Pub. No.: US 2020/0209260 A1
(43) Pub. Date: Jul. 2, 2020

(54) Early Diagnosis and Prognosis of Pre-Eclampsia

(71) Applicants: INSTITUT NATIONAL DE LA SANTE ET DE LA RECHERCHE MEDICALE (INSERM), Paris (FR); UNIVERSITE DE PARIS 5 RENE DESCARTES, Paris (FR); CENTRE NATIONAL DE LA RECHERCHE SCIENTIFIQUE, Paris (FR); INSTITUT NATIONAL DE LA RECHERCHE AGRONOMIQUE, Paris Cedex 07 (FR)

(72) Inventors: Daniel VAIMAN, Paris (FR); Jean-Luc VILOTTE, Paris (FR)

(21) Appl. No.: 16/339,298

(22) PCT Filed: Oct. 4, 2017
(86) PCT No.: PCT/FR2017/052728

§ 371 Date: Apr. 3, 2019

(30) Foreign Application Priority Data
Oct. 4, 2016 (FR) 1659555

(51) Int. Cl. G01N 33/68

(52) U.S. Cl. CPC G01N 33/6893; G01N 2800/52; G01N 2800/2857

(57) ABSTRACT

The invention relates to a process for the in vitro diagnosis or prognosis of pre-eclampsia, comprising determining an increase or decrease in the concentration of one or more markers in a sample of biological fluid.

### TECHNICAL FIELD

The invention relates to a novel process for the in vitro diagnosis or prognosis of pre-eclampsia.

### TECHNOLOGICAL BACKGROUND

Pre-eclampsia (PE) is a major disease of pregnancy, affecting 2 to 5% of pregnant women in industrialized countries. It is generally characterized by (i) abnormal arterial tension starting from the second trimester of gestation, with a systolic pressure of greater than 140 mm of mercury, and a diastolic pressure of greater than 90 mm of mercury, and (ii) proteinuria of greater than 300 mg per day. The disease appears at the earliest in the 20th week of pregnancy, but may be triggered later than this, for example after the 37th week, the normal duration of gestation in women being approximately 41 weeks. The consequences for the mother range from very moderate to fatal, insofar as pre-eclampsia may become complicated by cerebral (eclampsia), hepatic (HELLP syndrome) and/or vascular attacks. Of 830 000 births per year in France, approximately 40 000 present with pre-eclampsia, leading to death of the mother in approximately 10 cases per year. The consequences for the fetus are also significant, since treating the disease amounts to attempting to control the hypertension, but often requires the extraction of the fetal-placental unit, making pre-eclampsia a major contributor to iatrogenic prematurity. Moreover, approximately ⅓ of cases of pre-eclampsia become complicated by intrauterine growth restriction, without necessarily being associated with premature birth. It is estimated that approximately 500 children die each year in France due to the consequences of pre-eclampsia (extreme prematurity or birth weight too low).

Therapy for pre-eclampsia is highly limited, as is often the case for diseases affecting pregnant women. It is possible to use antihypertensives (for example nimodipine, hydralazine, labetalol, diazoxide, ketanserin), but all these molecules have drawbacks in their kinetics of action. Several studies are in agreement that a low dose of aspirin has a marginal, but beneficial, role;

however, it would appear that the effect of aspirin could be significant when it is administered very early, i.e. before 16 weeks of pregnancy (Bujold et al., Prevention of preeclampsia and intrauterine growth restriction with aspirin started in early pregnancy: a meta-analysis. Obstetrics and Gynecology 2010 August; 116, p 402-414). There is therefore a major therapeutic difficulty to be faced: symptoms appearing at the earliest in the second trimester of pregnancy, and a treatment which is potentially effective only if administered in the first trimester of pregnancy.

Seeking presymptomatic markers of pre-eclampsia has therefore become a priority in order to be able to give an early diagnosis or prognosis of pre-eclampsia, in order to be able to effectively treat the disease. The international effort has been considerable in this regard, and presymptomatic serum markers have been identified. These are, in particular, soluble VEGF receptor (sFLT1), soluble endoglin receptor (sENG), PAPP-A (pregnancy-associated plasma protein A) receptor and PIGF (Placental Growth Factor) receptor. The two former markers are increased in cases of pre-eclampsia, and the two latter are decreased. Some companies are already producing “predictive” kits (Alere, Roche), the effectiveness of which is far from perfect in terms of specificity and sensitivity in the early stages of pregnancy, especially before the 16th week. There is therefore a real need to identify new markers which enable early diagnosis and prognosis of pre-eclampsia, especially in the gestation period earlier than or equal to 16 weeks.

### SUMMARY OF THE INVENTION

The present invention aims to propose a novel method for the in vitro diagnosis or prognosis of pre-eclampsia, enabling very early detection of pre-eclampsia or very-early detection of a risk of developing the disease.

According to a first aspect, the invention relates to a method for the in vitro diagnosis or prognosis of pre-eclampsia, comprising:
a) measuring, in a sample of biological fluid originating from a pregnant woman, the concentration of one or more markers chosen from LIFR, TTR (transthyretin), ApoA2 and Pzp (A2M), preferably LIFR and/or TTR, alone or in combination with one or more other markers,
b) using the result of the measurement of step a) in the diagnosis or prognosis of pre-eclampsia, wherein a positive diagnosis or a positive prognosis is given by an increase or a decrease in the concentration of the marker(s) relative to the normal concentration of the marker(s) obtained in pregnant women.

According to a second aspect, the invention relates to the use of one or more markers in the in vitro evaluation of pre-eclampsia in a sample of biological fluid originating from a pregnant woman, wherein a positive diagnosis or a positive prognosis is given by an increase or a decrease in the concentration of the marker(s) relative to the normal concentration of the marker(s) obtained in pregnant women, said marker(s) being chosen from LIFR, TTR (transthyretin), ApoA2 and Pzp (A2M), preferably LIFR and/or TTR, alone or in combination with one or more other markers.

According to a third aspect, the invention relates to the use of one or more antibodies directed against markers in the in vitro evaluation of pre-eclampsia in a sample of biological fluid originating from a pregnant woman, wherein a positive diagnosis or a positive prognosis is given by an increase or a decrease in the concentration of the marker(s) relative to the normal concentration of the marker(s) obtained in pregnant women, said marker(s) being chosen from LIFR, TTR (transthyretin), ApoA2 and Pzp (A2M), preferably LIFR and/or TTR, alone or in combination with one or more other markers.

According to a fourth aspect, the invention relates to a diagnosis or prognosis kit of use for carrying out the process according to the invention.

According to a fifth aspect, the invention relates to a method for treating pre-eclampsia in a pregnant woman, comprising:
(i) carrying out a process according to the invention, and
(ii) when the diagnosis is positive or the prognosis is positive, introducing a suitable treatment for the pregnant woman.

### DETAILED DESCRIPTION OF THE INVENTION

### Definitions

For the purposes of the invention, the term “prognosis” denotes determining a probability, a risk or a possibility of developing pre-eclampsia in a pregnant woman. In particular, the pregnant woman is suspected of suffering from, or of being able to develop, pre-eclampsia.

For the purposes of the invention, the term “diagnosis” denotes determining a probability or a possibility of having pre-eclampsia in a pregnant woman. The term encompasses the early diagnosis (detection) of pre-eclampsia.

For the purposes of the invention, the term “biological fluid” denotes any fluid taken from a pregnant woman, from which is it possible to measure the concentration of the markers of the invention. For example, the biological fluid is chosen from blood, plasma, serum, urine, saliva or amniotic fluid, preferably serum. The biological fluid may be taken at any time during the pregnancy; the pregnant woman is preferably at a gestation period of less than or equal to 20 weeks, preferably less than or equal to 16 weeks, preferably at a gestation period of between 7 weeks and 16 weeks. This period, which corresponds to the first trimester of gestation, involves the preparation of significant vascular reorganization at the fetal-maternal interface, and it has been shown that effective treatment requires very early detection of the precursory signs of the disease.

LIFR (leukemia inhibitory factor receptor), also known under the name “CD118”, is the receptor for a cytokine (LIF) which acts on differentiation, survival and proliferation of a wide variety of cells. The measurement of LIFR in a sample of biological fluid may be carried out by conventional methods, for example by ELISA, using an antibody which binds to LIFR. LIFR exists in a soluble form and in a membrane-bound form. In the context of the present invention, the LIFR is soluble LIFR.

ApoA2 (apolipoprotein A-II) is a protein present in the plasma in monomeric, homodimeric or heterodimeric form with apolipoprotein D. The measurement of ApoA2 in a sample of biological fluid may be carried out by conventional methods, for example by ELISA, using an antibody which binds to ApoA2.

A2M (alpha-2-macroglobulin), also known under the name Pzp (pregnancy zone protein), is a plasma protein composed of four identical subunits bonded together by disulfide bridges. A2M also exists in dimeric and monomeric form. A2M is especially synthesized by the liver. The measurement of A2M in a sample of biological fluid may be carried out by conventional methods, for example by ELISA, using an antibody which binds to A2M.

Transthyretin (TTR) is a tetrametric protein present in plasma and cerebrospinal fluid which has four identical subunits. It is especially synthesized in the liver and in the choroid plexus. It constitutes one of the proteins that binds thyroxine (T4), a thyroid hormone, and vitamin A (retinol). It also has a role in neurogenesis, axon growth and nerve regeneration. Its role in pre-eclampsia has been described in the literature, where a decrease in the concentration of transthyretin has been shown in the serum of symptomatic patients at a late stage of the disease (Kalkunte et al. 2013, Transthyretin is dysregulated in preeclampsia, and its native form prevents the onset of disease in a preclinical mouse model, Am J Pathology, and Zhu et al., Exp Ther Med, 2014). The measurement of TTR in a sample of biological fluid may be carried out by conventional methods, for example by immunoprecipitation and MALDI (matrix-assisted laser desorption/ionization) (Théberge et al. 2000, Detection of transmethyretin variants using immunoprecipitation and matrix-assisted laser desorption/ionization bioactive probes: a clinical application of mass spectrometry, J Am Soc Mass Spectrom).

PAPP-A (pregnancy-associated plasma protein A) is a protein which has been described as having a role in pre-eclampsia (Dugoff et al. 2004 First-trimester maternal serum PAPP-A and free-beta subunit human chorionic gonadotropin concentrations and nuchal translucency are associated with obstetric complications: A population-based screening study (The FASTER Trial), Am J Obstet Gynecol). This protein has been discovered to be specific to the placenta and certain cancers; it is assumed that it could be an inhibitor of the complement cascade, with a proteolytic activity. A decrease in the serum concentration of PAPP-A is observed in cases of pre-eclampsia. It may for example be detected using a specific antibody in an ELISA assay.

sFLT1 (soluble fms-like tyrosine kinase-1) is a protein of the tyrosine kinase family which has been described as a marker of pre-eclampsia (Levine et al. (2006), Soluble endoglin and other circulating antiangiogenic factors in preeclampsia, NEJM). sFLT1 competes with membrane-bound receptors of VEGF, VEGFR 1, 2 and 3, preventing the cellular activation thereof and thus reducing angiogenesis, in particular placental angiogenesis. An increase in the serum concentration of sFLT1 is observed in cases of pre-eclampsia. sENG (endoglin) is a protein which has been presented as a marker of pre-eclampsia, with an increase in the serum concentration thereof in patients suffering from the disease. sENG acts as a soluble, and hence inactive, receptor of the signalling pathway of BMP (bone morphogenetic protein, from the TGFβ family), molecules with a pro-angiogenic impact. From this perspective, the role of sENG in pre-eclampsia would be highly comparable to that of the trio VEGF/sFLT1/VEGFRs.

PIGF (placental growth factor) is a protein which has also been presented as a marker of pre-eclampsia, with a decrease in the serum concentration thereof in patients suffering from the disease. It should be noted that PIGF also interacts with the VEGFR1 (flt1) receptor and is therefore a major contributor to active placental vascularization, which would explain the deleterious effect of the decrease thereof in cases of pre-eclampsia.

Fetal hemoglobin is one of the hemoglobins from the β hemoglobin family, produced by the fetus and the placenta. Fetal hemoglobin is found in the serum of pregnant women. Recent studies indicate that it increases early in pre-eclampsia patients (approximately doubled, yet with very great interindividual variation) (Anderson et al. Pregnancy Hypertension 6 (2016) 103-109).

Hemopexin is a protein which has also been presented as a marker of pre-eclampsia, with a decrease in the serum concentration thereof in patients suffering from the disease. The decrease in the concentration of hemopexin is proportional to the severity of the disease. Its essential biological role is that of eliminating hematin, a product of the degradation of extracellular heme. It has been known since 1978 that hemolytic disorders may be associated with pre-eclampsia. If the level thereof decreases, then the heme product will no longer be eliminated. The most recent study published shows a concentration of 1143 μg/ml in control patients, 947 in the most severe pre-eclampsia (p=0.04) and 1085 in more moderate pre-eclampsia (non-significant) (Anderson et al. Pregnancy Hypertension 6 (2016) 103-109).

The peptide sequences of the markers mentioned above are defined relative to the sequences currently listed in sequence databases at the priority date of the present application. Moreover, the specific sequences of the markers are examples. Those skilled in the art know that polymorphic variants may exist within the human population. These polymorphic variants generally only differ by a few amino acids (for example 1 to 5 or 1 to 3 amino acids).

For the purposes of the invention, the term “concentration” must be understood in its main sense, that is to say the amount of the entity of interest relative to a given volume. The concentration may for example be expressed as molar concentration (e.g. mol/l) or as mass concentration (e.g. g/l). The concentration of the marker(s) may be determined using any suitable method, preferably an immunological method. Methods for performing immunological analysis are well known to those skilled in the art, for example the ELISA method (enzyme-linked immunosorbent assay), the IRMA method (immunoradiometric assay), mass spectrometry, chromatography or the RIA method (radioimmunoassay). According to a preferred embodiment of the present invention, the concentration of the marker(s) is determined by ELISA. The normal concentration obtained in pregnant women, hereinafter the “normal concentration”, is determined conventionally, for example for the whole of the population or a specific population. The specific population may be defined for example based on ethnic origin or any other characteristic which may affect the normal concentration of the marker(s). The population for establishing the normal level of the markers is for example chosen based on a low risk of developing pre-eclampsia (that is to say without any history including a risk of pre-eclampsia, for example without prior pre-eclampsia, without diabetes or without hypertension). Once the normal concentrations are known, the determined concentrations of the marker(s) can be compared and the significance of the difference determined using standard statistical methods. The statistical methods make it possible especially to evaluate the specificity and sensitivity of the measurement. Mention may for example be made of ROC curves which make it possible to define a significance level. In a particular embodiment, if there is a significant difference between the determined concentration of the marker(s) and the normal concentration (i.e. a statistically significant difference), then there is a clinically significant probability that the pregnant woman has, or is at risk of developing, pre-eclampsia. The risk of developing pre-eclampsia will thus be quantified and expressed as probability using likelihood ratios. Determining the risk of developing pre-eclampsia may also make use of statistical parameters or algorithms widely known to those skilled in the art, such as the standard deviation score (Z score) for each marker.

For the purposes of the invention, the term “marker” (or “biomarker”) is a biological entity, for example a protein, RNA or DNA, which may be used for the diagnosis or prognosis of pre-eclampsia. For the purposes of the invention, the marker is preferably a protein. A protein is a biological macromolecule formed of one or more polypeptide chains, each of these chains consisting of amino acid residues bonded together by peptide bonds. In a preferred embodiment of the invention, the marker is in a soluble form.

### Process for Diagnosis or Prognosis

The present invention results from the surprising advantages demonstrated by the inventors, whereby an increase or decrease in the concentration of certain markers in a biological fluid enables a diagnosis or prognosis of pre-eclampsia. All the applications resulting therefrom are described below.

Indeed, the invention relates to a process for the in vitro diagnosis or prognosis of pre-eclampsia, comprising:
a) measuring, in a sample of biological fluid originating from a pregnant woman, the concentration of one or more markers chosen from LIFR, TTR (transthyretin), ApoA2 and Pzp (A2M), preferably LIFR and/or TTR, alone or in combination with one or more other markers,
b) using the result of the measurement of step a) in the diagnosis or prognosis of pre-eclampsia, wherein a positive diagnosis or a positive prognosis is given by an increase or a decrease in the concentration of the marker(s) relative to the normal concentration of the marker(s) obtained in pregnant women.

In a particular embodiment, the biological fluid is chosen from blood, plasma, serum, urine, saliva or amniotic fluid, preferably serum. The biological fluid may be analyzed immediately after the sample has been taken, or at a subsequent time. For example, the sample may be frozen or dried (e.g. lyophilized) then stored. The freezing or drying enables easy storage and subsequent analysis, while limiting the risk of altering the sample (for example denaturation of the proteins). The sample may also be stored with an agent which stabilizes the marker(s). The sample may also be prepared so as to increase the detectability of the marker(s), for example by fractionating and/or concentrating the sample.

As detailed above, one of the major advantages of the process according to the invention is that of enabling early diagnosis or prognosis of pre-eclampsia. Thus, in a particular embodiment, said pregnant woman is at a gestation period of less than or equal to 20 weeks, preferably less than or equal to 16 weeks, preferably at a gestation period of between 7 weeks and 16 weeks.

In a particular embodiment, a positive diagnosis or a positive prognosis is given by an increase or a decrease in the concentration of the marker(s) relative to the normal concentration of the marker(s) obtained in pregnant women. By way of example, a positive diagnosis or a prognosis may be in the form of a quantification of the risk of having, or developing, pre-eclampsia, for example expressed as percentage. By way of example, if there is a significant difference between the determined concentration of the marker(s) and the normal concentration obtained in pregnant women (i.e. a statistically significant difference), then there is a clinically significant risk that the pregnant woman has, or is at risk of developing, pre-eclampsia.

Some of the markers of the invention are present in the body in a circulating form and in a membrane-bound form (e.g. LIFR). In a preferred embodiment, the marker is in a circulating form (e.g. circulating LIFR or sLIFR; circulating TTR or sTTR; circulating ApoA2 or sApoA2; circulating Pzp or sPzp).

The process according to the invention may also be combined with other processes implementing other markers and/or measuring physiological parameters associated with (indicative of) pre-eclampsia. This may include, for example, (i) detecting the concentration of other markers, for example sENG, sFLT1, PIGF, PAPP-A, fetal hemoglobin and/or hemopexin, (ii) measuring certain physiological parameters, for example by measuring uterine or placental arterial velocity (e.g. Doppler ultrasound test), by the BOLD magnetic resonance imaging (MRI) test, and/or (iii) detecting markers for genetic predisposition, for example genes encoding at-risk HLA variants, genes encoding proteins of the coagulation cascade or genes encoding proteins of the complement cascade.

In a particular embodiment, said one or more other markers of step a) are chosen from sENG, sFLT1, PIGF, PAPP-A, fetal hemoglobin and/or hemopexin. In a particular embodiment, measuring the concentration of several other markers makes it possible to calculate ratios, for example the sFLT1/PIGF ratio.

In a particular embodiment of the present invention, measuring the concentration of one or more markers chosen from LIFR, TTR (transthyretin), ApoA2 and Pzp (A2M) makes it possible to calculate ratios, for example the LIFR/TTR, sFLT1/PIGF, sLIFR×sFLT1, LIFR/ApoA2 and/or LIFR/Pzp ratio. Thus, the present invention may comprise a step a′) of calculating one or more ratios of two markers and optionally calculating one or more ratios of two other markers, preferably calculating an sFLT1/PIGF and/or sLIFR×sFLT1 ratio. In this particular embodiment, step b) will therefore be: step b) using the result of the measurement of step a) and/or of the calculation of step a′) in the diagnosis or prognosis of pre-eclampsia, wherein a positive diagnosis or a positive prognosis is given by an increase or a decrease in (i) the concentration of the marker(s) and/or (ii) the ratio(s) of two markers relative to the normal concentration of the marker(s) obtained in pregnant women.

In certain embodiments, the process of the invention comprises a preliminary step of obtaining a sample of biological fluid from a pregnant woman. This step is upstream of the measurement of the concentration of the marker(s) in said sample of biological fluid.

As is widespread in the prior art, the concentration of the marker(s) may be standardized relative to the total protein concentration of the sample of biological fluid. Thus, the process according to the invention may comprise a step of determining the total protein concentration of the sample of biological fluid. This determining does not pose any particular difficulty and may be readily carried out by those skilled in the art by conventional methods for determining the total protein concentration.

The invention may also be defined as a process for the in vitro diagnosis or prognosis of pre-eclampsia, comprising determining an increase or a decrease in the concentration, relative to the normal concentration obtained in pregnant women, of one or more markers in a sample of biological fluid originating from a pregnant woman, said marker(s) being chosen from LIFR, TTR (transthyretin), ApoA2 and Pzp (A2M), preferably LIFR and/or TTR, alone or in combination with one or more other markers.

In a particular embodiment, a positive diagnosis or a positive prognosis is given by a concentration of the marker(s) that is greater than or less than the normal concentration of the marker(s) obtained in pregnant women. In a particular embodiment, the process also comprises determining an increase or a decrease in the concentration, relative to the normal concentration obtained in pregnant women, of one or more other markers in the sample of biological fluid, said other marker(s) being chosen from sENG, sFLT1, PIGF, PAPP-A, fetal hemoglobin and/or hemopexin.

### Uses

The invention also relates to the use of one or more markers in the in vitro evaluation of pre-eclampsia in a sample of biological fluid originating from a pregnant woman, wherein a positive diagnosis or a positive prognosis is given by an increase or a decrease in the concentration of the marker(s) relative to the normal concentration of the marker(s) obtained in pregnant women, said marker(s) being chosen from LIFR, TTR (transthyretin), ApoA2 and Pzp (A2M), preferably LIFR and/or TTR, alone or in combination with one or more other markers.

The invention also relates to the use of one or more antibodies directed against one or more markers in the in vitro evaluation of pre-eclampsia in a sample of biological fluid originating from a pregnant woman, wherein a positive diagnosis or a positive prognosis is given by an increase or a decrease in the concentration of the marker(s) relative to the normal concentration of the marker(s) obtained in pregnant women, said marker(s) being chosen from LIFR, TTR (transthyretin), ApoA2 and Pzp (A2M), preferably LIFR and/or TTR, alone or in combination with one or more other markers.

In a particular embodiment, a positive diagnosis or a positive prognosis is given by an increase or a decrease in one concentration ratios of two markers relative to the normal concentration of the marker(s) obtained in pregnant women, for example the LIFR/TTR, sFLT1/PIGF, sLIFR×sFLT1, LIFR/ApoA2 and/or LIFR/Pzp ratio, preferably the sFLT1/PIGF and/or sLIFR×sFLT1 ratio.

As it is used here, the term “antibody” refers to immunoglobulin molecules or other molecules which comprise at least one antigen-binding domain. It encompasses in particular whole antibodies, fragments of antibodies comprising an antigen-binding domain (e.g. Fab, Fab′ and F(ab)2, scFv, fragments comprising either a VL domain or a VH domain), monoclonal antibodies, polyclonal antibodies, chimeric antibodies, humanized antibodies, primatized antibodies, monospecific antibodies, multispecific antibodies, single-chain antibodies (e.g. of camelid type). The antibodies according to the invention may be molecules of any type, for example IgG, IgE, IgM, IgD, IgA and IgY, of any class, for example IgG1, IgG2, IgG3, IgG4, IgA1 and IgA2 or of any subclass.

Generally, polyclonal antibodies can be obtained by immunization of an animal with the marker in question (or a marker fragment), followed by recovery of the antibodies sought in purified form, by sampling the serum of said animal, and separation of said antibodies from the other constituents of the serum, especially by affinity chromatography on a column on which an antigen specifically recognized by the antibodies, especially said marker, is attached.

Generally, monoclonal antibodies can be obtained by the hybridoma technique, the general principle of which is recalled below. Firstly, an animal, generally a mouse, is immunized with the marker of interest (or a marker fragment), with the B lymphocytes of said animal then being capable of producing antibodies against said antigen. These antibody-producing lymphocytes are then fused with “immortal” myeloma cells (for example murine) in order to give hybridomas. Each hybridoma is multiplied in clone form, each one leading to the production of a monoclonal antibody, the properties of recognition of which, with regard to said marker, may be tested for example by ELISA, by immunoblotting (Western blot), either one-dimensional or two-dimensional, with immunofluorescence, or using a biosensor. The monoclonal antibodies selected in this way are subsequently purified, especially using the chromatography technique. The monoclonal antibodies may also be recombinant antibodies obtained by genetic engineering by techniques well-known to those skilled in the art.

In a preferred embodiment, the antibody or antibodies directed against one or more markers are monoclonal antibodies. Preferably, the monoclonal antibody or antibodies directed against one or more markers are used for measuring an increase or a decrease in the concentration in an ELISA assay.

### Treatment Method

The process according to the invention may also be used in order to monitor the effectiveness of a prophylactic treatment for preventing the development of pre-eclampsia, wherein a reduction in the risk of developing pre-eclampsia will be the indicator of the effectiveness of the prophylactic treatment. As described above, the process according to the invention is especially of use for the early diagnosis or prognosis of pre-eclampsia, which makes it possible both to provide early treatment to patients in order for them to receive suitable care, and also to not treat patients who do not require treatment.

Thus, the present invention also relates to a method for treating pre-eclampsia in a pregnant woman, comprising:
(i) carrying out a process of diagnosis or prognosis according to the invention, and
(ii) when the diagnosis is positive or the prognosis is positive, introducing a suitable treatment for the pregnant woman.

In a particular embodiment, said suitable treatment is one or more medicaments chosen from aspirin (acetylsalicylic acid), magnesium sulfate, antihypertensives, corticosteroids, low molecular weight heparins, cholesterol regulators (e.g. pravastatin) and oxidative stress regulators (e.g. bound to circulating heme, such as alpha-1-microglobulin), preferably aspirin.

### Kits

The invention also relates to diagnosis or prognosis kits of use for carrying out the processes according to the invention. The kits generally comprise one or more reagents (for example one or more antibodies which bind to the marker(s) of interest) making it possible to determine the concentration of the marker(s). The kits may also comprise one or more containers and/or one or more tubes suitable for mixing the sample of biological fluid with the reagent(s). In some embodiments, the kits may comprise one or more surfaces which have an affinity for the marker(s) which may be brought into contact with the sample of biological fluid (for example one or more ELISA plates). The kits may contain one or more suitable washing solutions. In some embodiments, the kits may also comprise other components, such as one or more buffers, one or more preservatives or one or more protein stabilizers. The kit may also comprise one or more components necessary for the detection of the marker(s) (for example an enzyme and/or a substrate).

### FIGURE LEGENDS

FIG. 1: diagram depicting the results of the iTRAQ experiment showing the increase or the decrease in the concentration of several markers present in the plasma of mice having pre-eclamptic gestation relative to the normal concentration of these markers in the plasma of mice having normal gestation, at 6.5 days of gestation (the total gestation for the mice line studies is 18.5 days). Among the markers studied with a modified concentration in the pre-eclamptic mice which are useable in humans due to gene homology, it may be noted that the concentrations of the proteins LIFR and TTR are highly modified. It should be noted that the family A serpins (Serpina #) are not readily transposable to humans due to significant polygenism of this gene in mice.

FIG. 2: diagram depicting the concentration of circulating LIFR in the plasma of 30 pre-eclamptic women and 30 control women, taken at the time of labour (A. crude measurement, and B. relative to total proteins). The pre-eclamptic women have a significantly higher concentration of circulating LIFR in their plasma than the control women.

FIG. 3: diagram depicting the concentration of circulating LIFR and sFLT1 in the plasma of 20 pre-eclamptic patients and 20 control patients, as crude measurement or relative to the total proteins. The pre-eclamptic women have a significantly higher concentration of circulating LIFR in their plasma than the control women. The results show that the LIFR marker appears to be more discriminating than the sFLT1 marker, current reference marker.

FIG. 4: curve depicting the concentration of circulating LIFR in the plasma of 20 pre-eclamptic patients and 20 control patients, as crude measurement as a function of the gestation date. It is noted that, regardless of the gestation stage, the concentration of circulating LIFR is increased in the plasma of pre-eclamptic patients compared to the concentration of circulating LIFR in control patients. The control patients are defined as pregnant patients in good health, without any known medical complication (no diabetes mellitus, pre-existing hypertension, renal or cardiovascular pathology, no twin pregnancy or other multiple pregnancy).

FIG. 5: diagram depicting the concentration of PIGF and sENG (markers already known) in first trimester plasma in 100 patients who later developed pre-eclampsia and 50 control patients.

FIG. 6: diagram depicting the concentration TTR in first trimester plasma in 100 patients who later developed pre-eclampsia and 50 control patients.

FIG. 7: diagram depicting the concentration sFLT1 (known marker) in first trimester plasma in 100 patients who later developed pre-eclampsia and 50 control patients.

FIG. 8: diagram depicting the concentration sLIFR in first trimester plasma in 100 patients who later developed pre-eclampsia and 50 control patients.

FIG. 9: ROC curve depicting the effectiveness of the ratio of sFLT1/PIGF concentration in first trimester plasma in 100 patients who later developed pre-eclampsia and 50 control patients. The closer the area under the curve is to 1, and the more different it is to 0.5, and therefore the greater the area under the curve (ROC area), the more effective is the marker.

FIG. 10: diagram and curve depicting the ratio of sLIFR×sFLT1 concentration in first trimester plasma in 100 patients who later developed pre-eclampsia and 50 control patients. The closer the area under the curve is to 1, and the more different it is to 0.5, and therefore the greater the area under the curve (ROC area), the more effective is the marker.

### EXAMPLES

### Example 1: Preparation of the Murine Model of Pre-Eclampsia by Overexpression of STOX1

The cDNA of the complete open reading frame (ORF) of the human STOX1 gene (isoform A) was microinjected into a male pronucleus following fertilization. The pronucleus transfected in this way was implanted into the uterus of a female mouse. After 18.5 days of gestation, transgenic mice were obtained. The number of copies of the transgene STOX1 was evaluated by quantitative polymerase chain reaction (qPCR) using the DNA from the transgenic mouse relative to a single-copy gene.

Three transgenic mouse lines were obtained and amplified. The analysis of the transmission of the STOX1 transgene through the generations showed that there was a single insertion locus of the transgene STOX1 over the 3 transgenic mouse lines which were amplified. The phenotyping of the transgenic mice was carried out as described in Doridot et aL, Hypertension, 2013, with daily measurement of the blood pressure throughout gestation, and urine sampling for measuring proteinuria. In the article by Doridot et al., it was also shown that markers characteristic of pre-eclampsia (sFLT1 and sENG) are increased in the serum/plasma of transgenic mice carrying fetuses and which are thus pre-eclamptic.

### Example 2: Demonstrating the Markers of the Invention in Mice

Pooled or isolated plasma samples were collected from heparinized blood from the jugular vein of transgenic mice of example 1, carrying fetuses, at 6.5 days of gestation (pre-eclamptic mice) or non-pre-eclamptic mice at 6.5 days of gestation (control group). These plasmas were depleted in the major plasma proteins on MARS-3 columns (Agilent), making it possible to eliminate albumin, IgGs and transferrin. For this purpose the plasmas were diluted 20-fold in the equilibration buffer provided with the MARS-3 column, then filtered by microcentrifugation over 0.22 μm filters. Two washes at 100 g for 2.5 min were carried out, and the fraction obtained was concentrated using a 5K MWCO concentrator. The amount of remaining proteins was evaluated by 2D Quant protein assay (GE Healthcare, France). The fraction was subsequently aliquoted and stored at −80° C. Thereafter, samples containing 50 μg of proteins were lyophilized, and then resuspended in 25 μl of 500 mM TEAB buffer (Sigma) with 1% SDS. The proteins contained in the samples were then alkylated, digested and labelled with the iTRAQ reagents according to the manufacturer's protocols (Applied Biosystems) in order to obtained labelled peptides. The samples containing the labelled peptides are hereinafter referred to as “labelled samples”. The labelled samples were identified as a function of their status: pre-eclampsia or control. The labelled samples were then combined and lyophilized. In order to limit complexity, the labelled peptides (hereinafter “peptides”) were washed and fractionated by chromatography on a cation exchange column. 200 μg of dry peptides were resuspended in 1 ml of 5 mM KH₂PO₄ and 20% acetonitrile (Carlo Erba), pH 2.8 (buffer A). 80% of the peptides were pre-fractionated with a 5 μm PolySULFOETHYL A column (PolyLC, Columbia, Md.) 200 mm long×2.1 mm in diameter and with a pore size of 300 Å, on an HPLC (Waters625 HPLC) with a constant flow of 2 ml/min. The column was first washed with the buffer A from the manufacturer for 30 min, then a gradient of 19 min was applied to increase the concentration of buffer B to 63% then a plateau of 5 min and again 100% of buffer A for 5 min. Fractions were collected every 30 sec, pooled as a function of the UV absorbance at 210 nm (16 μg/pool). For the extraction at high salt concentration, desalination was carried out with a C18-Sep-Pak cartridge (Waters). The fractions were subsequently lyophilized and stored at −20° C. The fractions were then resuspended in 0.1% TFA buffer (Fluka) and 10% acetonitrile (Carlo Erba), then 2 μg were injected in two goes into a nano-HPLC Ultimate 3000 (Dionex). The peptides were then purified using a C18 PepMap column (Dionex, 0.3 mm I.D.×5 mm, pore size 100 Å, 3 μm particles) at 30 μl/min in 0.1% TFA (Fluka) and 2% acetonitrile (Carlo Erba). The peptides were then separated with a C18 PepMap 100 column (Dionex, 75 mm I.D.×150 mm, pore size 100 Å, 3 μm particles) at 300 nl/min (solution A: 0.1% TFA, 2% acetonitrile, solution B: 20% solution A mixed vol/vol with 80% acetonitrile). After equilibration with 7% of B, a multi-step gradient is started 3 min after injection, with 16% of B 14 min post-injection, 19% at 22 min, 23% at 25 min, 32% at 51 min, 50% at 65 min and a plateau at 95% for 79 min before equilibration for 16 min. Fractionation was then carried out for each 2 μg duplicate with a Dionex robot (Probot), 18 min after the start of the injection, and the collection was coupled directly with MALDI plates, every 10 seconds for 384 points per fraction. The eluent and the matrix solution were mixed then placed on the MALDI plates. The matrix of α-cyano-4-hydrocinnamic acid (CHCA) was dissolved at 2 mg/ml in a solution of acetonitrile (70%) with 0.1% TFA and 110 μM of glu-fibrinopeptide-B for internal calibration (m/z=1570.677).

The mass spectra were measured with a 4800 MALDI-TOF-TOF spectrometer (Applied Biosystems), version 3.5.28193, build 1011 with a positive reflection mode at a fixed laser frequency. For each fraction, 50 spectra were collected between 850 and 4000 Da with a laser frequency fixed at 200 Hz. 500 spectra per sample were summed and processed to obtain monoisotopic values with a raw spectrum, the signal/noise ratio of which was greater than 20. Each spectrum made it possible to select the 8 most abundant peaks with a signal/noise ratio >15. 1000 MS/MS spectra were summed for each precursor with the baseline subtracted using the Savitsky-Golay algorithm (4th degree polynomial regression). In order to identify the non-dominant proteins, an exclusion list was produced from a duplicated series.

The peptides were identified with ProteinPilot version 2.0.1 (Applied Biosystems, MDS-Sciex, Foster City, Calif.). The analysis was made by comparison with the ‘Human Protein Database’ of the IPI (International protein Index version 3.38). The data was processed taking into account the trypsin cleavage carried out of the carbamidomethylated cysteines, with the search strength set at “thorough”. A protein is considered to be significantly identified when at least two definitely identified peptides are assigned a confidence score >95% and a degree of difference on the iTRAQ of >1.2, with p<0.05.

The results are presented in table 1 below.

TABLE 1
list of markers exhibiting an increase in the concentration or a decrease in the
concentration in transgenic mice relative to the normal concentration of the markers
obtained in wild-type mice (control group or “WT”), at 6.5 days of gestation.

| Name of | | Number of | Tg/WT at |
|---|---|---|---|
| marker | Full name | peptides | 6.5 days |
| Gm20547 | Protein Gm20547 OS = Mus musculus | 3 | 1.34 |
| | GN = Gm20547 PE = 2 SV = 1 | | |
| LIFR | Putative uncharacterized protein OS = Mus musculus | 44 | 1.24 |
| | GN = Lifr PE = 2 SV = 1 | | |
| Serpina1b | Serpina1b protein OS = Mus musculus | 9 | 1.24 |
| | GN = Serpina1b PE = 2 SV = 1 | | |
| Hrg | Histidine-rich glycoprotein OS = Mus musculus | 8 | 1.10 |
| | GN = Hrg PE = 1 SV = 2 | | |
| Kng1 | Kininogen-1 OS = Mus musculus | 39 | 1.08 |
| | GN = Kng1 PE = 1 SV = 1 | | |
| Serpina1a | Alpha-1-antitrypsin 1-1 OS = Mus musculus | 6 | 1.07 |
| | GN = Serpina1a PE = 1 SV = 4 | | |
| F2 | Coagulation factor II OS = Mus musculus | 16 | 1.05 |
| | GN = F2 PE = 2 SV = 1 | | |
| Serpina6 | Serine (Or cysteine) peptidase inhibitor, | 28 | 0.97 |
| | clade A, member 6 OS = Mus musculus | | |
| | GN = Serpina6 PE = 2 SV = 1 | | |
| Cycs | Cytochrome c OS = Mus musculus | 1 | 0.97 |
| | GN = Cycs PE = 2 SV = 1 | | |
| Ces1c | Carboxylesterase 1C OS = Mus musculus | 51 | 0.88 |
| | GN = Ces1c PE = 1 SV = 4 | | |
| Cp | Ceruloplasmin OS = Mus musculus | 63 | 0.88 |
| | GN = Cp PE = 2 SV = 1 | | |
| Pzp(A2M) | Alpha-2-macroglobulin OS = Mus musculus | 45 | 0.85 |
| | GN = Pzp PE = 2 SV = 1 | | |
| Kng2 | Kng2 protein OS = Mus musculus | 5 | 0.82 |
| | GN = Kng2 PE = 2 SV = 1 | | |
| Apoa2 | APOAII OS = Mus musculus | 25 | 0.79 |
| | GN = Apoa2 PE = 4 SV = 1 | | |
| Obp1a | MCG117626 OS = Mus musculus | 10 | 0.76 |
| | GN = Obp1a PE = 2 SV = 2 | | |
| TTR | Transthyretin OS = Mus musculus | 18 | 0.54 |
| | GN = Ttr PE = 2 SV = 1 | | |

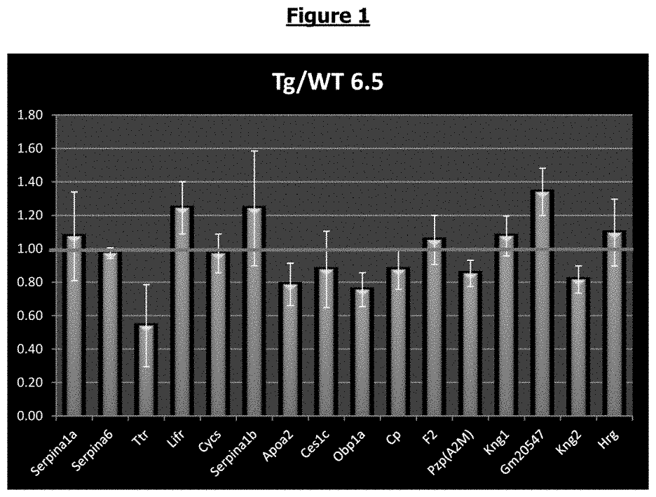

The “number of peptides” column indicates the number of peptides which were clearly identified in the protein during the iTRAQ. The more peptides that are identified, the more is accurate the identification of the corresponding protein. The number of peptides therefore indicates the reliability of the identification of the protein, and hence of the measurement. The correct protein can be considered to have been identified beyond 2 peptides identified in the same protein. The overall results at 6.5 days are presented in FIG. 1.

### CONCLUSION

The concentration of the markers presented in table 1 is either increased or decreased in mice with pre-eclampsia. These markers may therefore be used within the context of the invention.

The markers having a large number of peptides and an increase or a decrease in the concentration at least equal to 20% relative to the wild-type mice are TTY (transthyretin), LIFR, ApoA2 and Obp1a. LIFR appears to be a particularly beneficial marker for the in vitro diagnosis or prognosis of pre-eclampsia.

### Example 3: Study of the LIFR Marker in Pregnant Women at 25 Weeks of Pregnancy

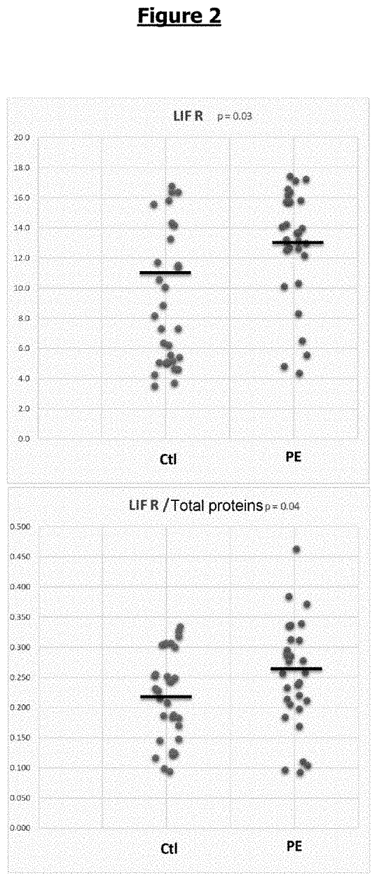

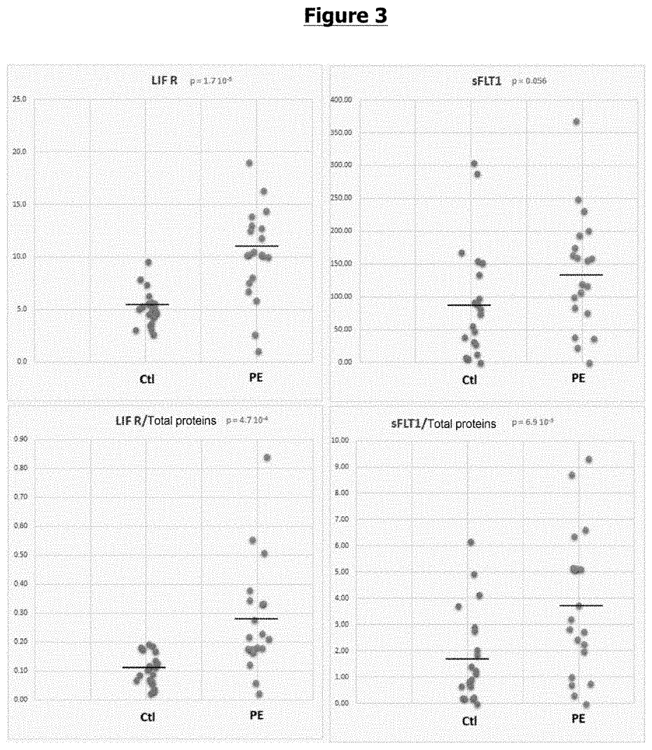

The concentration of LIFR in the plasma of pregnant women at 25 weeks of pregnancy was measured for FIG. 2 and FIG. 3 with the BOSTER kit (reference EK1200), precisely following the manufacturer's technical recommendations. The detail may be obtained at www.bosterbio.com. The protocol is standardized to operate in strips constituting a 96-well plate. The kit provides a standard which makes it possible to produce a response range by successive dilutions, making it possible to calibrate the unknown samples. After addition of the final solution, a more or less intense yellow staining was read in absorbance at 450 nm. The results were integrated into the standard range (from 156 pg/ml to 10 ng/ml) by linear regression. More precisely, the diluted LIFR standard was prepared 2 hours before the experiment by consecutive dilutions in a diluent provided in the kit. The range was deposited in duplicates in 16 wells (8 concentrations) of the 96-well plate provided. The 80 other samples were dilutions of human samples from serum from pregnant women, either pre-eclamptic or not. The plate was subsequently placed for 1 h 30 at 37° C. After evacuating the supernatant, 100 μl of the anti-LIFR antibody from the BOSTER kit (reference EK1200), diluted in the diluent provided in the kit, were added. The plate was then incubated for 1 h at 37° C. The wells are then rinsed 3 times with 300 μl of PBS. 100 μl per well of the ABC solution (Avidin-Biotin-Peroxidase Complex), diluted in the dilution buffer provided, were subsequently added. Incubation was carried out at 37° C. for 30 minutes. The wells were rinsed 5 times with PBS, then 90 μl of TMB buffer were added (containing the peroxidase substrate). Incubation was carried out at 37° C. for 30 minutes, then the TMB stop solution was added (100 μl). The absorbance could subsequently be measured at 450 nm with a spectrophotometer.

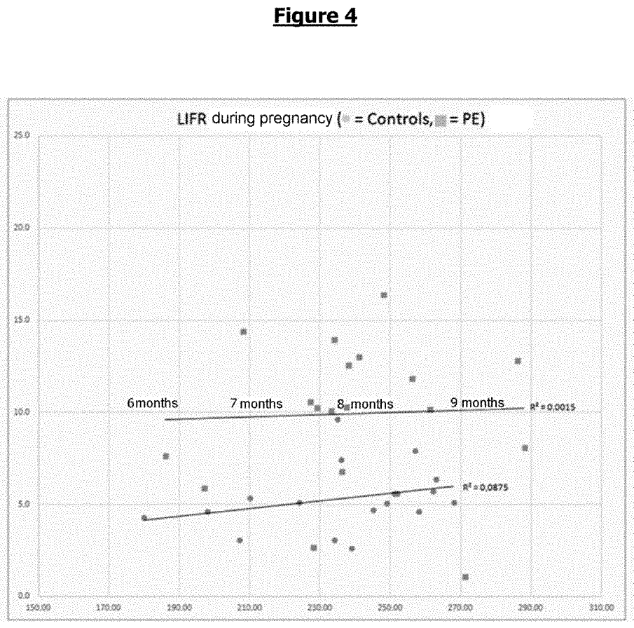

The amount of sFLT1a was measured for FIGS. 3 and 4 with the kit sold by MyBioSource under the catalogue number MBS175839, precisely following the manufacturer's technical recommendations. The detail may be obtained at http://www.mybiosource.com/. The protocol is identical to that presented for the LIFR measurement. The protocol is standardized to operate in strips constituting a 96-well plate. The kit provides a standard which makes it possible to produce a response range by successive dilutions, making it possible to calibrate the unknown samples. After addition of the final solution, a more or less intense yellow staining was read in absorbance at 450 nm. The results were integrated into the standard range by linear regression.

### LIFR Marker

The results are presented in FIGS. 2, 3 and 4.

### Conclusion for the First Cohort (FIG. 2):

The mean concentration of LIFR detected in the plasma of pregnant women was 11.1 ng/ml in the controls (pregnant women without PE), as opposed to 12.9 ng/ml in the PE patients (unilateral T test, 0.028, on paired cases 0.023). The results confirm that the concentration of the LIFR marker increases in the plasma of pregnant women with PE.

Conclusion for the Second Cohort and Comparison with sFLT1 (FIGS. 3 and 4):

Highly significant differences for the concentration of sLIFR were observed between the PE patients and the controls (mean=5.1 ng/ml in the controls, 10.6 ng/ml in the PE patients (p=1.7 10⁻⁵)). The reference marker sFLT1 was also tested on the same plasma samples; in this case, the difference was, respectively, 92 ng/ml and 135 ng/ml in the controls and the PE patients (p=0.056).

FIG. 3 presents the standardization of the LIFR concentration relative to the total protein amount, calculated by the Bradford colorimetric assay relative to a range of bovine serum albumin. We obtained a significant result (1.7 ng/ml vs. 3.8 ng/ml, p=0.007). On the earliest samples (4 controls and 3 PE of 6-7 months, i.e. 25-30 weeks), the controls had a lower concentration of LIFR than the PE patients.

The data suggests that sLIFR has better performance than sFLT1 in discriminating between a sample originating from a PE patient or a control. In addition, despite the limited number of early samples (around 25 weeks), the differences observed are just as large early in the pregnancy as late in the pregnancy (FIG. 4).

### Example 4: Studies of the Markers LIFR, TTR, sFLT1, PAPP-A, PIGF and of the sFLTt1/PIGF Ratio in Pregnant Women Around 13-14 Weeks of Gestation

Plasma samples taken around 13-14 weeks of gestation from pregnant women who went on to develop PE were recovered. These samples are systematically analyzed by a plate ELISA method for all the markers sought: LIFR (BOSTER kit), TTR (Abcam kit), sFLT1 (R&D kit), PAPP-A (Thermofisher kit) and PLGF (R&D kit). As a reminder, the standard ELISA measurement protocol is as follows. The kit provides a standard which makes it possible to produce a response range by successive dilutions, making it possible to calibrate the unknown samples. After addition of the final solution, a more or less intense yellow staining can be read in absorbance at 450 nm. The results are integrated into the standard range (from 156 pg/ml to 10 ng/ml) by linear regression.

The marker concentration is deduced by implementing similar protocols, detailed for each of the kits. For example, for the LIFR marker, the diluted LIFR standard is prepared 2 hours before the experiment by consecutive dilutions in a diluent provided in the kit. The range is deposited in duplicates in 16 wells (8 concentrations) of the 96-well plate provided. The 80 other samples are a dilution of the human samples. The plate is subsequently placed for 1 h 30 at 37° C. After evacuating the supernatant, 100 μl of the anti-LIFR antibody, diluted in the diluent provided in the kit, are added. The plate is then incubated for 1 h at 37° C. 100 μl per well of the ABC solution (Avidin-Biotin-Peroxidase Complex), diluted in the dilution buffer provided, are subsequently added. Incubation is carried out at 37° C. for 30 minutes. The wells are rinsed 5 times with PBS, then 90 μl of TMB buffer are added (containing the peroxidase substrate). Incubation is carried out at 37° C. for 30 minutes, then the TMB stop solution is added (100 μl). The absorbance can subsequently be measured at 450 nm with a spectrophotometer. The protein concentrations will be deduced by linear regression or power regression relative to the ranges provided.

### Example 5: Studies of the Markers LIFR, TTR, sFLT1, sENG, PIGF, of the sFLTt1/PIGF and sLIFR×sFLT Ratios in Pregnant Women During the First Trimester of Pregnancy (<12 Weeks)

### Materials and Methods

Human samples: 150 samples of human plasma were obtained in collaboration with Prof. Stefan Hansson's team (Lund, Sweden). The samples were collected during the first trimester of pregnancy from patients after signing of informed consent at the hospital of Lund. After analysis, 50 control samples and 100 samples from patients suffering from pre-eclampsia were included in the study.

### Biochemical Analysis

The plasma samples were diluted in a diluent compatible with the ELISA technique in a 96-well microtitration plate. The sFLT1, sLIFR, sENG, PIGF and TTR ELISA kits were purchased respectively from Origene, BosterBio, NetBiotech, Cohesion Biosciences and NetBiotech. The ELISA assays were carried out according to the manufacturer's general recommendations as detailed below.

For each ELISA assay, 10 μl of plasma were diluted in 90 μl of diluent, except for the TTR ELISA for which the plasma samples were diluted to 1/10000, then the diluted samples were analyzed on 96-well plates in which antibodies capable of recognizing the protein of interest (sFLT1, sLIFR, sENG, PIGF or TTR) are fixed, for 1 h 30 to 2 h at 37° C. The liquid was subsequently removed from the wells and a secondary antibody capable of recognizing the protein of interest was added (100 μl, diluted to 1/100) then incubated for 1 h at 37° C. The samples were then washed 3-4 times with 300 μl of 0.01× PBS or a specific washing buffer. After each washing step, the liquid was removed and the plate turned over on a piece of absorbent paper, while avoiding the samples drying out. Then, a second anti-Fc antibody coupled to HRP (horseradish peroxidase) was added into the wells (100 μl, diluted to 1/100) then the plate was incubated for 30 min at 37° C. The samples were subsequently washed again with 0.01× PBS or a specific washing buffer 5 times, and on each washing the liquid was removed and the plate turned over on a piece of absorbent paper. Finally, the HRP substrate was added into the wells (90 μl), which led to blue staining proportional to the amount of protein of interest present in the sample. In parallel, 2×8 wells were used to prepare standard samples for each of the proteins of interest, which served as corresponding calibration ranges (from 10 ng to 156 pg of protein of interest, and one blank). Finally, an acid stop solution (50 μl or 100 μl, depending on the instructions in the user's manual) was added into each of the wells, which led to a color change from blue to yellow of proportional intensity. The plate was subsequently automatically read by a plate reader (Berthold) set to 450 nm. The results were stored in an Excel file.

### Mathematical Analysis

A calibration curve was produced by regression analysis (linear or non-linear to obtain the optimal adjustment evaluated by the coefficient of determination, R²) from standard samples, then the concentrations of protein of interest of each plasma sample were estimated by applying the regression curve and taking into account the dilution factor. The results of the analysis of the plasma samples were then analyzed by parametric tests (Student's t-tests) and non-parametric tests (Mann-Whitney), using Excel's Statist'XL software. In addition, the grouping of the markers was analyzed using a hierarchical tree classification tool from Statist'XL from the inter-marker correlation matrix. The ROC curves were calculated with the software developed by John Eng on the website www.rad.jhmi.edu/jeng/javarad/roc/JROCFITi html.

### Results

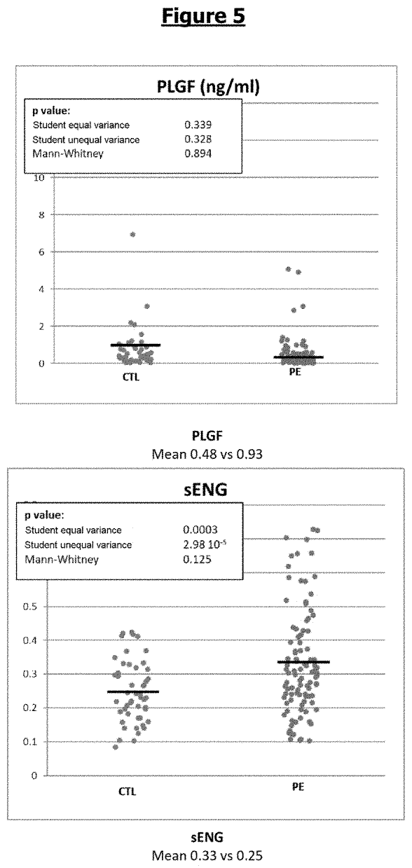

The results are presented in FIGS. 5 to 10.

FIG. 5: the markers PIGF and sENG (markers of the prior art) were tested on the 150 samples of human plasma. The concentration of the PIGF marker is decreased in the PE (pre-eclampsia) samples relative to the control (CTL) samples. The concentration of the sENG marker is increased in the PE (pre-eclampsia) samples relative to the control (CTL) samples. These results are therefore consistent with that which has already been described in the literature for the markers PIGF and sENG. On the other hand, the markers PIGF and sENG for diagnosing pre-eclampsia did not appear to give statistically significant differences. Nonetheless, one sample appears to be problematic (outlier) in the CTL cohort (control). By removing this sample, the diagnosis of pre-eclampsia with the sENG marker becomes statistically significant.

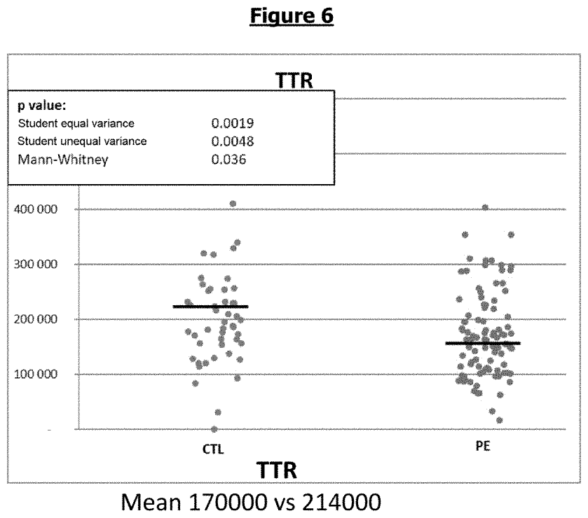

FIG. 6: the concentration of the TTR marker is significantly decreased in parametric tests and non-parametric tests in the PE (pre-eclampsia) samples relative to the control (CTL) samples. However, we observed overlaps.

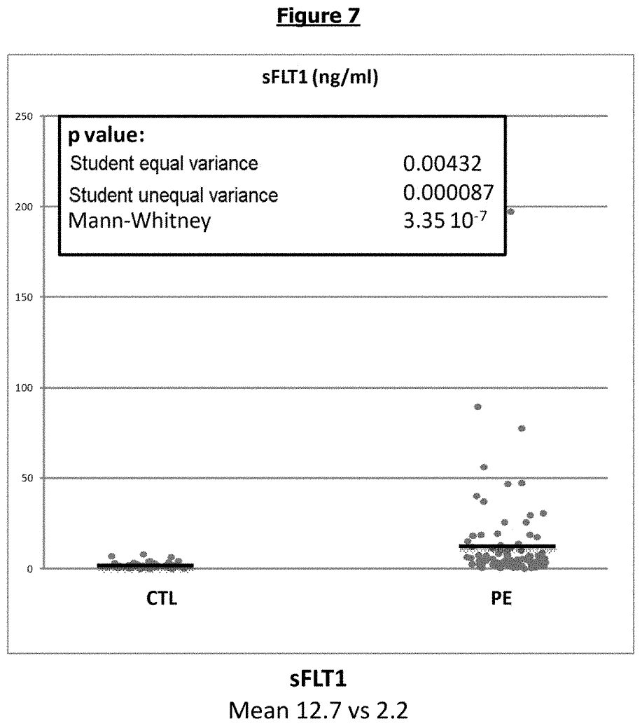

FIG. 7: the concentration of the sFLT1 marker is significantly increased in parametric tests and non-parametric tests in the PE (pre-eclampsia) samples relative to the control (CTL) samples. This marker gave excellent results in the detection of pre-eclampsia with a mean factor of 6-fold more in the disease cases.

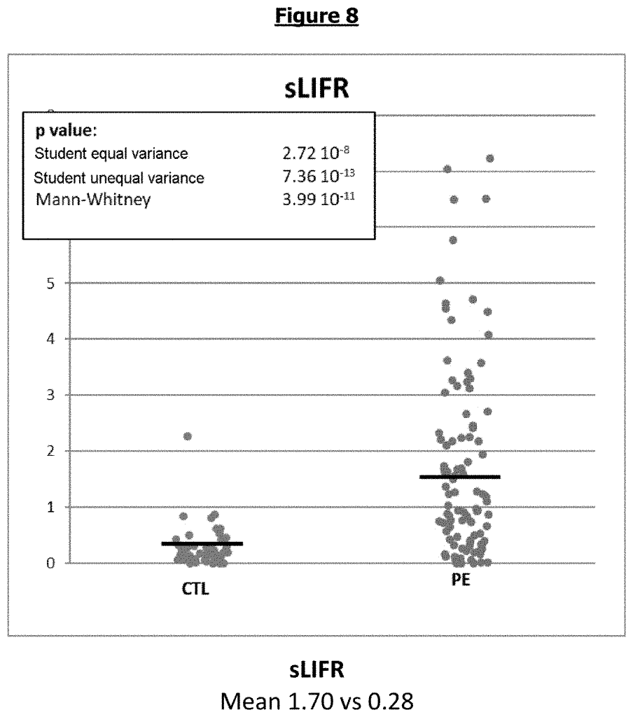

FIG. 8: the concentration of the sLIFR marker is significantly increased in parametric tests and non-parametric tests in the PE (pre-eclampsia) samples relative to the control (CTL) samples. This marker proved to have extremely good performance in the detection of pre-eclampsia, in particular after elimination of the control sample behaving as an outlier (the same as for sENG and PIGF). The mean difference was of the order of 6-fold.

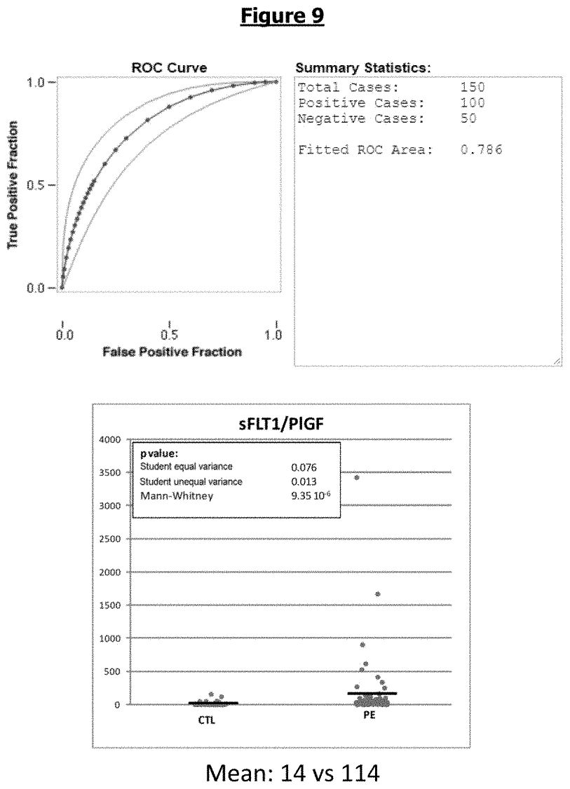

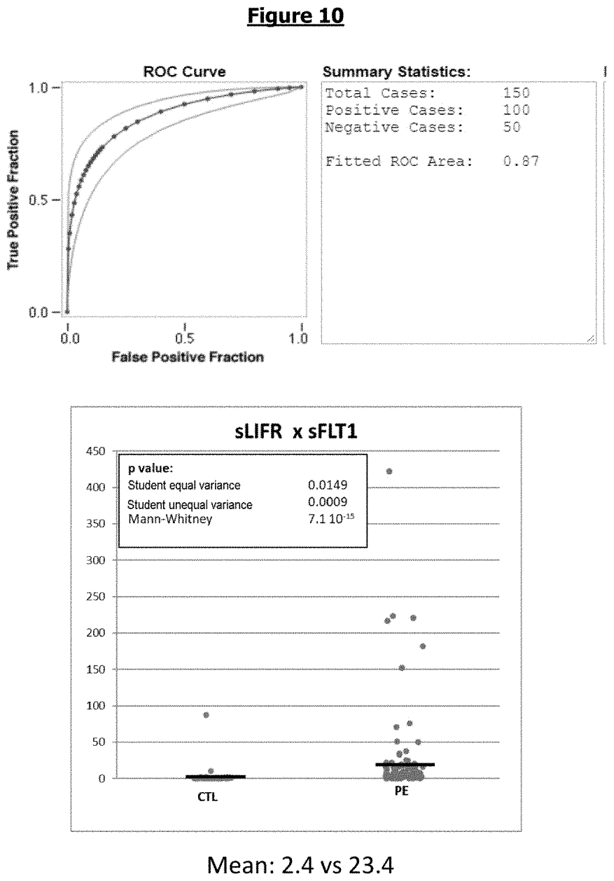

FIGS. 9 and 10: the curves combining two markers show an excellent result of the “synthetic” markers sFLT1/PIGF and sFLT1×sLIFR. For example, values of the order of 10-fold greater in the PE group, and a very good predictive power of the occurrence of pre-eclampsia for sFLT1×sLIFR. The area under the ROC curve (0.87) was maximum relative to the isolated markers or in other combinations.

1. A process for the in vitro diagnosis or prognosis of pre-eclampsia, comprising:
a) measuring, in a sample of biological fluid originating from a pregnant woman, the concentration of one or more markers chosen from LIFR, TTR (transthyretin), ApoA2 and Pzp (A2M), preferably LIFR and/or TTR, alone or in combination with one or more other markers,
b) using the result of the measurement of step a) in the diagnosis or prognosis of pre-eclampsia, wherein a positive diagnosis or a positive prognosis is given by an increase or a decrease in the concentration of the marker(s) relative to the normal concentration of the marker(s) obtained in pregnant women.

2. The process as claimed in claim 1, said biological fluid is chosen from blood, plasma, serum, urine, saliva or amniotic fluid, preferably serum.

3. The process as claimed in either one of claims 1 and 2, said pregnant woman being at a gestation period of less than or equal to 20 weeks, preferably less than or equal to 16 weeks, preferably at a gestation period of between 7 weeks and 16 weeks.

4. The process as claimed in any one of claims 1 to 3, said marker being in a circulating form (sLIFR, sTTR, sApoA2 and sPzp).

5. The process as claimed in any one of claims 1 to 4, said one or more other markers being chosen from sENG, sFLT1, PIGF, PAPP-A, fetal hemoglobin and/or hemopexin.

6. The process as claimed in any one of claims 1 to 5, comprising a step a′) of calculating one or more ratios of two markers and optionally calculating one or more ratios of two other markers, preferably calculating the LIFR/TTR, sFLT1/PIGF and/or sLIFR×sFLT1 ratio.

7. The use of one or more markers in the in vitro evaluation of pre-eclampsia in a sample of biological fluid originating from a pregnant woman, wherein a positive diagnosis or a positive prognosis is given by an increase or a decrease in the concentration of the marker(s) relative to the normal concentration of the marker(s) obtained in pregnant women, said marker(s) being chosen from LIFR, TTR (transthyretin), ApoA2 and Pzp (A2M), preferably LIFR and/or TTR, alone or in combination with one or more other markers.

8. The use of one or more antibodies directed against one or more markers in the in vitro evaluation of pre-eclampsia in a sample of biological fluid originating from a pregnant woman, wherein a positive diagnosis or a positive prognosis is given by an increase or a decrease in the concentration of the marker(s) relative to the normal concentration of the marker(s) obtained in pregnant women, said marker(s) being chosen from LIFR, TTR (transthyretin), ApoA2 and Pzp (A2M), preferably LIFR and/or TTR, alone or in combination with one or more other markers.

9. The use as claimed in either one of claims 7 and 8, said one or more other markers being chosen from sENG, sFLT1, PIGF, PAPP-A, fetal hemoglobin and/or hemopexin.

10. A diagnosis or prognosis kit of use for carrying out the process as claimed in one of claims 1 to 6.

11. A method for treating pre-eclampsia in a pregnant woman, comprising:
(i) carrying out the process as claimed in any one of claims 1 to 6, and
(ii) when the diagnosis is positive or the prognosis is positive, introducing a suitable treatment for the pregnant woman.

12. The method as claimed in claim 11, said suitable treatment being one or more medicaments chosen from aspirin (acetylsalicylic acid), magnesium sulfate, antihypertensives, corticosteroids, low molecular weight heparins, cholesterol regulators and oxidative stress regulators, preferably aspirin.
